(19) United States
(12) Patent Application Publication
(10) Pub. No.: US 2018/0310651 A1
(43) Pub. Date: Nov. 1, 2018

(54) Therapeutic Energy Patch

(71) Applicant: Doyle Dianko Boyd, JR., South Richmond, NY (US)

(72) Inventor: Doyle Dianko Boyd, JR., South Richmond, NY (US)

(21) Appl. No.: 15/965,837

(22) Filed: Apr. 27, 2018

Related U.S. Application Data
(60) Provisional application No. 62/491,128, filed on Apr. 27, 2017.

(51) Int. Cl. A41D 27/10; A61K 33/00; A61K 33/16

(52) U.S. Cl. CPC A41D 27/10; A61K 33/00; A61K 33/16; A42B 1/24; A41D 2400/62; A41D 2400/32; A41D 2300/32

(57) ABSTRACT

A therapeutic energy patch is an apparatus that is mounted onto a user's clothes and transfers therapeutic energy into the user's body. The apparatus includes an apparel, a patch, and a quantity of gemstones. The patch allows the apparatus to restrain the quantity of gemstones onto the apparel. The quantity of gemstones generates the therapeutic energy, which radiates into the user's body.

The current application claims a priority to the U.S. Provisional Patent application Ser. No. 62/491,128 filed on Apr. 27, 2017.

### FIELD OF THE INVENTION

The present invention relates generally to an addition to clothing or apparel. More specifically, the present invention uses embedded crystalline materials to provide the user with a variety of health and athletic performance benefits, including generally enhanced healing and recovery ability in the area proximal to the patch attachment site.

### BACKGROUND OF THE INVENTION

Crystals and the like have long been utilized for their medicinal properties. Onyx has been linked to absorption and transformation of negative energy and has stress-relief effects. Tiger's eye is a mineral known to improve willpower and vitality, among other mindful effects. Clear quartz is associated with clarity of thought, and thus with improved mental performance. The properties of these and similar minerals have been demonstrated to result in overall positive changes and effects on the human body and have been demonstrated to be non-harmful with respect to effects on the skin.

These energetic effects are often utilized by medical technicians and practitioners; however, they are not utilized to their maximum potential. Often, the effects of crystal enhancement can be achieved through simple contact with the skin in an affected area. Some recent inventions even make use of the healing properties of certain minerals as a means of improving athletic performance and recovery. However, even among the products that make use of crystal technology, there is a lack of versatility. The products are limited to specific applications or uses and are therefore limited. What is needed is a device that can be applied to a variety of apparel and clothing, such that contact is maintained with the skin of the user.

The present invention addresses these issues. The present invention is a device that can be attached to daily-use apparel, such as hats, book bags, purses, shirts, pants, and shoes; athletic wear including jerseys, compression sleeves, leg and arm bands; and even jewelry, including necklaces and bracelets. By attaching the present invention in such a way as to facilitate contact with the user's skin, the user receives the benefits of a myriad of health benefits resulting from contact with included beneficial minerals. In patch form, the present invention is easy to place on existing clothing to improve usefulness and functionality.

### BRIEF DESCRIPTION OF THE DRAWINGS

FIG. 1 is an illustration of the present invention being worn a user's arm.

FIG. 2 is a schematic view of the present invention utilizing one patch.

FIG. 3 is a schematic view of the present invention utilizing two patches.

### DETAILED DESCRIPTION OF THE INVENTION

All illustrations of the drawings are for the purpose of describing selected versions of the present invention and are not intended to limit the scope of the present invention.

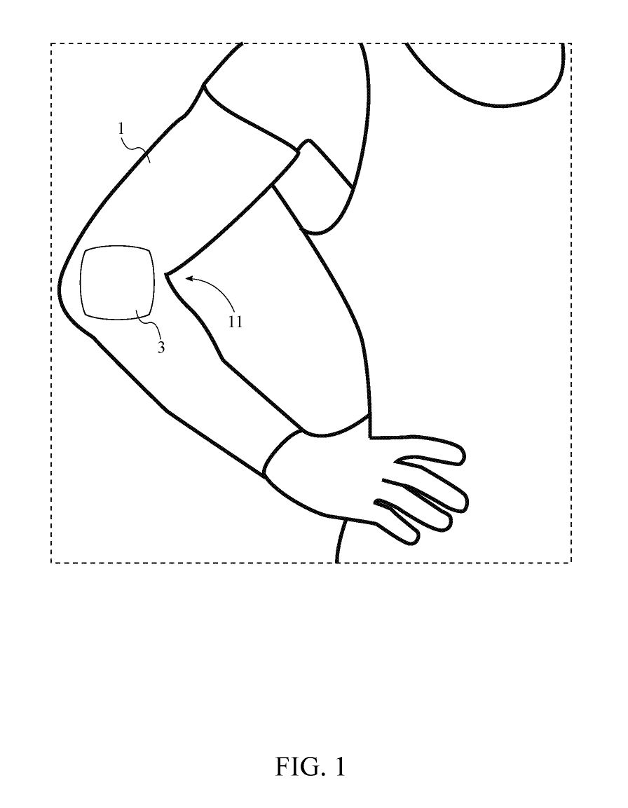

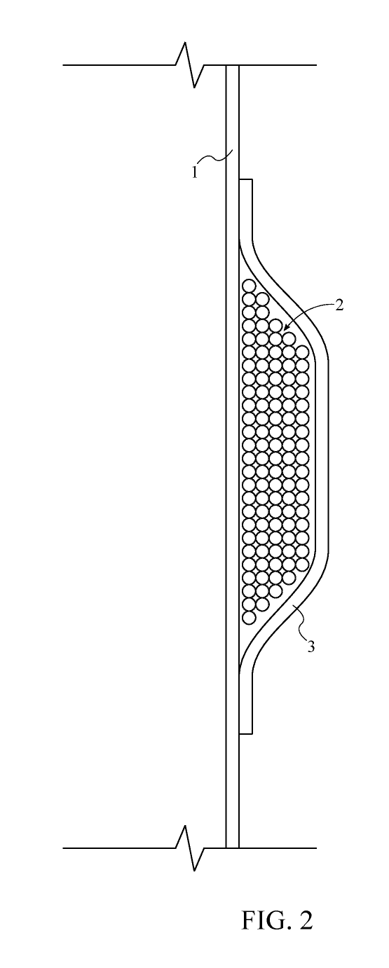

The present invention is a therapeutic energy patch that is retrofitted or integrated into a piece of clothing, which comes into contact with a user's skin or hair. The proximal positioning of the present invention to the user's skin or hair allows the present invention to transfer therapeutic energy into the user's body, which improves physical and mental healing for the user's body. The present invention comprises an apparel 1, a quantity of gemstones 2, and a first patch 3, which are shown in FIGS. 1 and 2. The apparel 1 is any piece of clothing that can be worn a user. The apparel 1 is preferably an athletic compression sleeve that can be worn on one of the user's appendages (e.g. a leg sleeve or an arm sleeve). However, the apparel 1 can alternatively be, but is not limited to, a hat, a shirt, or a pair of pants. The quantity of gemstones 2 radiates advantageous healing-related effects for the user. The preferred stone type for the quantity of gemstones 2 is citrine. However, the stone type for the quantity of gemstones 2 can also be, but is not limited to, smoky quartz, fluorite, clear quartz, carnelian, onyx, or tiger's eye. Alternatively, the quantity of gemstones 2 can be a mixture of any of the aforementioned stone types. The first patch 3 allows the present invention to restrain the quantity of gemstones 2 against the apparel 1. The first patch 3 is preferably made of a moisture-wicking fabric because the present invention is catered towards athletic clothing.

As can be seen in FIGS. 1 and 2, the general configuration of the aforementioned components allows the present invention to efficiently and effectively transfer the therapeutic energy in the user's body. The first patch 3 is externally positioned on the apparel 1, which prevents the excess bulge created by the present invention from uncomfortably pressing against the user's skin or hair. The first patch 3 is also perimetrically mounted to a specific section 11 of the apparel 1 in order to create a confined space in between the first patch 3 and the apparel 1. The specific section 11 of the apparel 1 can be arbitrarily chosen or can have a preferred location. If the apparel 1 is catered towards athletic clothing, then the specific section 11 of the apparel 1 is preferably a joint-bracing section of the apparel 1 so that the present invention can address the typical issue of joint pain with athletes. For example, the first patch 3 would be positioned adjacent to the elbow-bracing portion of the apparel 1 or the knee-bracing portion of the apparel 1. Moreover, the confined space in between the first patch 3 and the specific section 11 of the apparel 1 houses the quantity of gemstones 2, which keeps the quantity of gemstones 2 properly secured to the user's body.

The quantity of gemstones 2 can be configured into a variety of forms, which depend on what scenario the present invention is implemented by the user. In one scenario, if the apparel 1 is a headwear (e.g. a hat or a visor), then the quantity of gemstones 2 can be configured into a granular form. The granular form of the gemstones against the user's head will not become a nuisance to the user because a person's head stays relatively in place. In another scenario, if the apparel 1 is a piece of athletic clothing, then the quantity of gemstones 2 can be configured into a powdered form. The powdered form of the gemstones against the user's body will not become a nuisance as the user more athletically moves their body during a sport, exercise, or other physical activity.

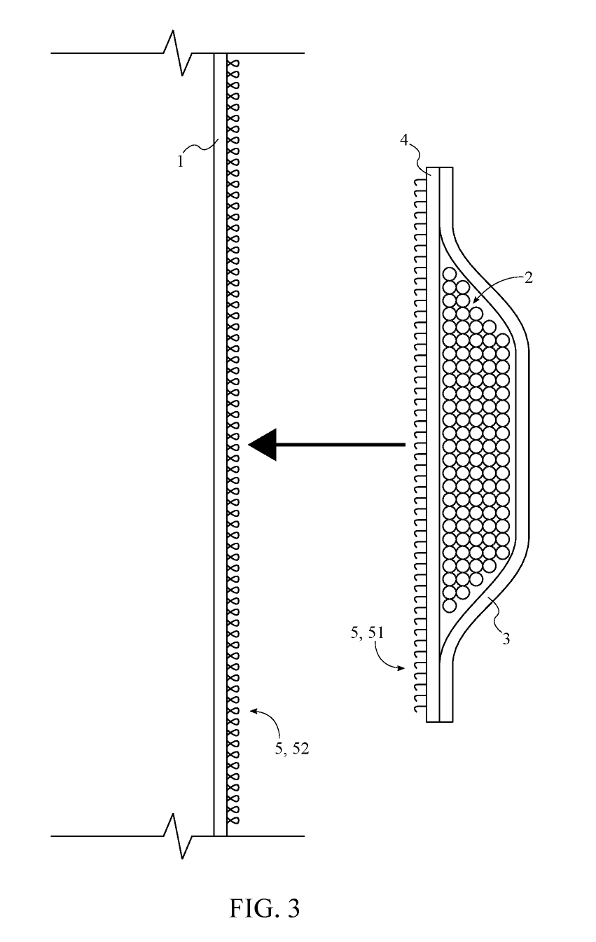

The quantity of gemstones 2 could be restrained on the apparel 1 by the first patch 3 in two different ways. In one way illustrated in FIG. 2, the first patch 3 is directly mounted onto the specific section 11 of the apparel 1 so that the confined space is delineated in between the first patch 3 and the specific section 11 of the apparel 1. This direct mounting of the first patch 3 provides the confined space with a permanent positioning on the apparel 1. In another way illustrated in FIG. 3, the present invention further comprises a second patch 4 and a fastening mechanism 5, which allow the first patch 3 to be indirectly mounted onto the specific section 11 of the apparel 1. Similar to the first patch 3, the second patch 4 is preferably made of a moisture-wicking fabric because the present invention is catered towards athletic clothing. More specifically, the first patch 3 is perimetrically connected onto the second patch 4 so that the confined space is delineated in between the first patch 3 and the second patch 4. Consequently, the quantity of gemstones 2 is housed in between the first patch 3 and the second patch 4. The fastening mechanism 5 allows the user to detach the first patch 3 and the second patch 4 from the specific section 11 of the apparel 1 and to move the quantity of gemstones 2 to another section of the apparel 1. Thus, the second patch 4 is attached onto the specific section 11 of the apparel 1 by the fastening mechanism 5.

More specifically for the other way, the fastening mechanism 5 is preferably a hook-and-loop fastener, which comprises a first interlocking piece 51 and a second interlocking piece 52. The first interlocking piece 51 is the hooks portion of the hook-and-loop fastener, and the second interlocking piece 52 is the loops portion of the hook-and-loop fastener, or vice versa. The first interlocking piece 51 is connected on the second patch 4, opposite the first patch 3, which allows the quantity of gemstones 2 to readily attach onto the apparel 1. The second interlocking piece 52 is positioned external to the apparel 1 and is integrated across the apparel 1, which allows the quantity of gemstones 2 to attach anywhere onto the apparel 1. The first interlocking piece 51 is releasably engaged to the second interlocking piece 52, which holds the quantity of gemstones 2 in place onto a selected section of the apparel 1.

### Alternate Description:

The present invention is an energy vibration patch that enables improved recovery and healing, both physically and mentally. The present invention is a patch that can be attached to clothing, such that the patch and contained minerals remain in contact with the user's skin or hair. The present invention comprises a patch and a connection means.

The patch is a preferably flexible unit that enables containment of crystalline and mineral materials, and simple attachment of the present invention to a variety of clothing and apparel. The patch comprises a first side, a second side, a chamber, and a plurality of minerals. The first side is a flat, generally flexible segment of the patch that, in the preferred arrangement of the present invention, enables attachment of the present invention to the inside of an item of clothing or apparel. The second side is a flat, generally flexible, permeable segment of the patch opposite the first side that, in the preferred arrangement of the present invention, contacts the user's skin, hair, or other external features. The first side and the second side attach at the edges to form the chamber. The chamber is a space that enables containment of the plurality of minerals. The plurality of minerals is a set of minerals, crystalline or otherwise, and elements that combine to create advantageous effects for a user. In an exemplary embodiment, the plurality of minerals comprises any or any combination of onyx, tiger's eye, clear quartz, and more. In an alternative embodiment, the plurality of minerals is a mesh pattern of threads of any or any combination of onyx, tiger's eye, clear quartz, and more, interwoven to create a synergistic, restoring and healing effect.

The connection means is any of a variety of permanent connectors that joins the patch to the user's clothing or apparel. The connection means may be any or any combination of stitching, sewing, hemming, adhesives, and more connectors. The connection means ensures connection of the first side of the patch flush with the user's clothing or apparel.

In the preferred usage of the present invention, the user acquires the present invention. The user places the first side of the patch against a piece of clothing or unit of apparel. The user then joins the present invention to the piece of clothing or unit of apparel and applies the connection means to the first side, combining the patch to the piece of clothing or unit of apparel. The user then benefits from constant contact of the plurality of minerals of the patch through the permeable second side, for as long as the patch is in contact with the user's skin, hair, or body. In an exemplary usage of the present invention, the user applies the patch to an elbow band. The patch relieves the elbow joint of pain during activity or helps the user to recover after stress has been placed on the relevant joint. In an embodiment, the user may remove the present invention from the piece of clothing or unit of apparel and place the patch upon another piece of clothing or unit of apparel to achieve similar mineral effects on other areas of the body. The present invention is prepared for subsequent application.

Although the invention has been explained in relation to its preferred embodiment, it is to be understood that many other possible modifications and variations can be made without departing from the spirit and scope of the invention as hereinafter claimed.

What is claimed is:

1. A therapeutic energy patch comprises:
an apparel;
a quantity of gemstones;
a first patch;
the first patch being externally positioned on the apparel;
the first patch being perimetrically mounted to a specific section of the apparel; and
the quantity of gemstones being housed in between the first patch and the specific section of the apparel.

2. The therapeutic energy patch as claimed in claim 1, wherein the quantity of gemstones is configured into a powdered form.

3. The therapeutic energy patch as claimed in claim 1, wherein the first patch is made of a moisture-wicking fabric.

4. The therapeutic energy patch as claimed in claim 1, wherein the specific section of the apparel is a joint-bracing section of the apparel.

5. The therapeutic energy patch as claimed in claim 1, wherein the apparel is an athletic compression sleeve.

6. The therapeutic energy patch as claimed in claim 1 comprises:
a second patch;
a fastening mechanism;
the first patch being perimetrically connected to the second patch;
the quantity of gemstones being housed in between the first patch and the second patch; and
the second patch being attached onto the specific section of the apparel by the fastening mechanism.

7. The therapeutic energy patch as claimed in claim 5 comprises:
the fastening mechanism being a hook-and-loop fastener;
the fastening mechanism comprises a first interlocking piece and a second interlocking piece;
the first interlocking piece being connected onto the second patch, opposite the first patch;
the second interlocking piece being positioned external to the apparel;
the second interlocking piece being integrated across the apparel; and
the first interlocking piece being releasably engaged to the second interlocking piece.

8. The therapeutic energy patch as claimed in claim 5, wherein the second patch is made of a moisture-wicking fabric.

9. The therapeutic energy patch as claimed in claim 1, wherein the quantity of gemstones is a stone type selected from a group consisting of: citrine, smoky quartz, fluorite, clear quartz, carnelian, onyx, tiger's eye, and combinations thereof.

10. A therapeutic energy patch comprises:
an apparel;
a quantity of gemstones;
a first patch;
the first patch being externally positioned on the apparel;
the first patch being perimetrically mounted to a specific section of the apparel;
the quantity of gemstones being housed in between the first patch and the specific section of the apparel;
the quantity of gemstones is configured into a powdered form;
the first patch being made of a moisture-wicking fabric;
the specific section of the apparel being a joint-bracing section of the apparel; and
the apparel being an athletic compression sleeve.

11. The therapeutic energy patch as claimed in claim 10 comprises:
a second patch;
a fastening mechanism;
the first patch being perimetrically connected to the second patch;
the quantity of gemstones being housed in between the first patch and the second patch; and
the second patch being attached onto the specific section of the apparel by the fastening mechanism.

12. The therapeutic energy patch as claimed in claim 11 comprises:
the fastening mechanism being a hook-and-loop fastener;
the fastening mechanism comprises a first interlocking piece and a second interlocking piece;
the first interlocking piece being connected onto the second patch, opposite the first patch;
the second interlocking piece being positioned external to the apparel;
the second interlocking piece being integrated across the apparel; and
the first interlocking piece being releasably engaged to the second interlocking piece.

13. The therapeutic energy patch as claimed in claim 11, wherein the second patch is made of a moisture-wicking fabric.

14. The therapeutic energy patch as claimed in claim 10, wherein the quantity of gemstones is a stone type selected from a group consisting of: citrine, smoky quartz, fluorite, clear quartz, carnelian, onyx, tiger's eye, and combinations thereof.

15. A therapeutic energy patch comprises:
an apparel;
a quantity of gemstones;
a first patch;
a second patch;
a fastening mechanism;
the fastening mechanism being a hook-and-loop fastener;
the fastening mechanism comprises a first interlocking piece and a second interlocking piece;
the first patch being externally positioned on the apparel;
the first patch being perimetrically mounted to a specific section of the apparel;
the quantity of gemstones being housed in between the first patch and the specific section of the apparel;
the first patch being perimetrically connected to the second patch;
the quantity of gemstones being housed in between the first patch and the second patch;
the second patch being attached onto the specific section of the apparel by the fastening mechanism;
the first interlocking piece being connected onto the second patch, opposite the first patch;
the second interlocking piece being positioned external to the apparel;
the second interlocking piece being integrated across the apparel; and
the first interlocking piece being releasably engaged to the second interlocking piece.

16. The therapeutic energy patch as claimed in claim 15, wherein the quantity of gemstones is configured into a powdered form.

17. The therapeutic energy patch as claimed in claim 15, wherein the first patch is made of a moisture-wicking fabric, and wherein the second patch is made of a moisture-wicking fabric.

18. The therapeutic energy patch as claimed in claim 15, wherein the specific section of the apparel is a joint-bracing section of the apparel.

19. The therapeutic energy patch as claimed in claim 15, wherein the apparel is an athletic compression sleeve.

20. The therapeutic energy patch as claimed in claim 15, wherein the quantity of gemstones is a stone type selected from a group consisting of: citrine, smoky quartz, fluorite, clear quartz, carnelian, onyx, tiger's eye, and combinations thereof.
